(19) United States
(12) Patent Application Publication
(10) Pub. No.: US 2020/0297078 A1
(43) Pub. Date: Sep. 24, 2020

(54) SHOE COMPONENTS BASED ON CUSTOMER DATA

(71) Applicant: HEWLETT-PACKARD DEVELOPMENT COMPANY, L.P., Houston, TX (US)

(72) Inventors: Matthew G. LOPEZ, San Diego, CA (US); Edward PONOMAREV, San Diego, CA (US); Stephen G. MILLER, San Diego, CA (US); David Bradley SHORT, San Diego, CA (US); Steven H. SMITH, San Diego, CA (US); Friso HAGMAN, Paal (BE); Jempi WILSSENS, Paal (BE); Christian DECKER, Achim (DE)

(73) Assignee: HEWLETT-PACKARD DEVELOPMENT COMPANY, L.P., Houston, TX (US)

(21) Appl. No.: 16/088,505

(22) PCT Filed: Sep. 6, 2017
(86) PCT No.: PCT/US2017/050246

§ 371 Date: Sep. 26, 2018

Related U.S. Application Data
(60) Provisional application No. 62/385,375, filed on Sep. 9, 2016.

(51) Int. Cl. A43D 119/00; G06Q 50/04; G06F 30/10; A43D 3/02; G01L 5/16; A61B 5/103

(52) U.S. Cl. CPC A43D 119/00; G06Q 50/04; G06F 30/10; G06F 2113/10; G01L 5/16; A61B 5/1036; A43D 3/02

(57) ABSTRACT

An example physical last includes a body having a bottom region. The physical last also includes a fitting attachable to the bottom region to define a component of a shoe. The fitting is manufactured based on three dimensional (3D) scan data of a foot of a customer and pressure data associated with the foot of the customer.

### BACKGROUND

Shoes may be manufactured according to industry standard sizes for mass production. Using industry standard sizes is likely to accommodate a high percentage of the customer base.

### BRIEF DESCRIPTION OF THE DRAWINGS

Some examples of the present application are described with respect to the following figures:

FIG. 1 illustrates a system to manufacture a component of a shoe based on customer data, according to an example;

FIG. 2A-2C illustrate examples a physical last that receives a shaping device generated based on customer data to manufacture a component of a shoe;

FIG. 3 illustrates a shaping device that customizes a physical last based on customer data to manufacture a component of a shoe, according to an example;

FIG. 4 illustrates a programmable physical last to manufacture a component of a shoe based on customer data, according to an example;

FIG. 5 illustrates an injection molding process to be used with a physical last to manufacture a component of a shoe based on customer data, according to an example;

FIG. 6 illustrates a computing device that determines customizations of a physical last based on customer data, according to an example; and

FIG. 7 illustrates a computing device that determines customizations of a component of a shoe based on customer data, according to an example.

### DETAILED DESCRIPTION

Variations in foot sizes (e.g., length and width) exist among individual customers. Thus, a customer's foot size may not match well with an industry standard size, leading to an uncomfortable fit of the shoe.

Examples described herein provide a system to manufacture a customized component of shoe based on customer data. For example, a shaping device may be attached to a physical last to define a component of shoe for customization. The component may be manufactured using the last. The shaping device may be a fitting that is generated based on three dimensional (3D) scan data of a foot of a customer and pressure data associated with the foot of the customer. In another example, a shaping device may encompass a region of a physical last. The shaping device may include a set of actuators to customize the last by defining a shape of the region based on 3D scan data of a foot of a customer and pressure data associated with the foot of the customer. In another example, a set of actuators may be located within a physical last. A deployment of the set of actuators to define a shape of a region of the last may be based on 3D scan data of a foot of a customer and pressure data associated with the foot of the customer. Examples described herein may increase efficiency of manufacturing customized shoe.

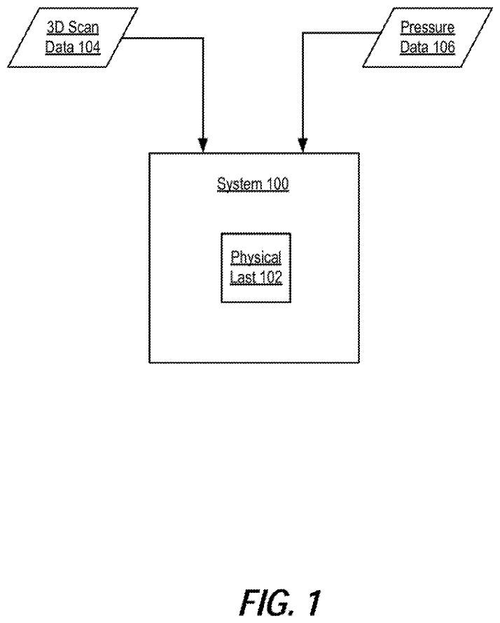

FIG. 1 illustrates a system 100 to manufacture a component of a shoe based on customer data, according to an example. System 100 may be a device or a set of devices that manufacture a component of shoe using a physical last. As used herein, a component of shoe may include an insole of a shoe, a midsole of a shoe, an upper of a shoe, or a combination thereof. An insole may be an inner layer of the sole of a shoe. A midsole may be a layer of the sole of the shoe that is between the insole and an outsole. An upper may be a portion of a shoe that holds the shoe to the foot of a customer.

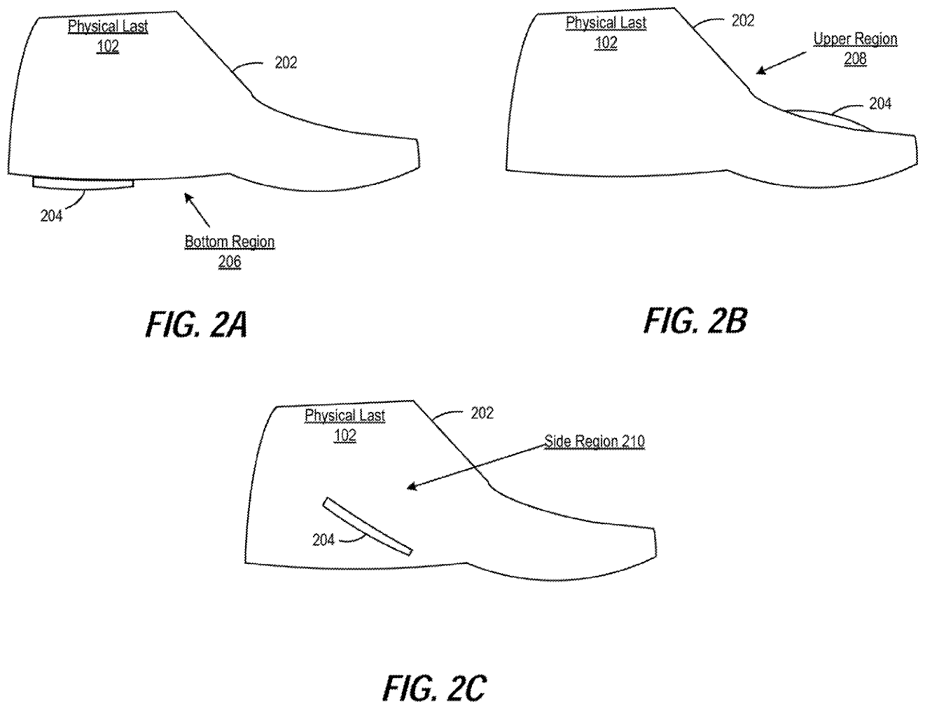

System 100 may include a physical last 102. Physical last 102 may be a physical device that is in the shape of a human foot. During operation, system 100 may receive three-dimensional (3D) scan data 104 of a foot of a customer and/or pressure data 106 associated with the foot of the customer. 3D scan data 104 may be measurements of the foot obtained using a 3D foot scanner. 3D scan data 104 may be used to create a 3D digital representation of the foot. Pressure data 106 may indicate pressure distribution experienced by the foot. Pressure data 106 may include both static pressure data (e.g., when the customer is standing still) and dynamic pressure data (e.g., when the customer is in motion). Pressure data 104 may be obtained using a pressure sensor. Based on 3D scan data 104 and pressure data 106, physical last 102 may be modified so that a component of a shoe manufactured using physical last 102 is customized for the particular customer. Modifying physical last 102 to manufacture a customized component of a shoe for a particular customer is described in more detail in FIGS. 2A-4.

FIG. 2A illustrates a physical last that receives a shaping device generated based on customer data to manufacture a component of a shoe, according to an example. Physical last 102 may include a housing 202. A region of housing 202 may receive a shaping device 204 to define a shape of the region so that a component of a shoe manufactured using physical last 102 with shaping device 204 is customized for a customer.

In an example, shaping device 204 may be a piece of fitting generated based on 3D scan data 104 and/or pressure data 106. Shaping device 204 may be manufactured in a plurality of manners. In an example, a digital representation of shaping device 204 may be determined from 3D scan data 104 and/or pressure data 106. The digital representation of shaping device 204 may include dimensions of shaping device 204 (e.g., length, width, depth, height, etc.). The digital representation of shaping device 204 may be used to manufacture shaping device 204 via a subtractive manufacturing process, an additive manufacturing process, a molding process, or a combination thereof.

In some examples, an additive manufacturing process may include a 3D printing process. The digital representation of shaping device 204 may be sent to a 3D printer for manufacturing. In some examples, in a subtractive manufacturing process, shaping device 204 may be first manufactured having standard dimensions (e.g., via an additive manufacturing process). Shaping device 204 may be cut or trimmed to custom dimensions based on the digital representation of shaping device 204 in a milling machine. In some examples, the digital representation of shaping device 204 may be used to create a mold for injection molding. The mold may be used to create shaping device 204.

Once manufactured, shaping device 204 may be attached to a bottom region 206 of housing 202. For example, shaping device 204 may be attached to bottom region 206 via magnets, clips, screws, etc. When shaping device 204 is attached to bottom region 206, housing 202 with shaping device 204 may be used to create a component of a shoe that is customized for a particular customer.

Shaping device 204 may also be attached to other regions of housing 202, which is described in more detail in FIGS. 2B-2C. As illustrates in FIG. 2B, shaping device 204 may be attached to an upper region 208 of housing 202. As illustrates in FIG. 2C, shaping device 204 may be attached to a side region 210 of housing 202.

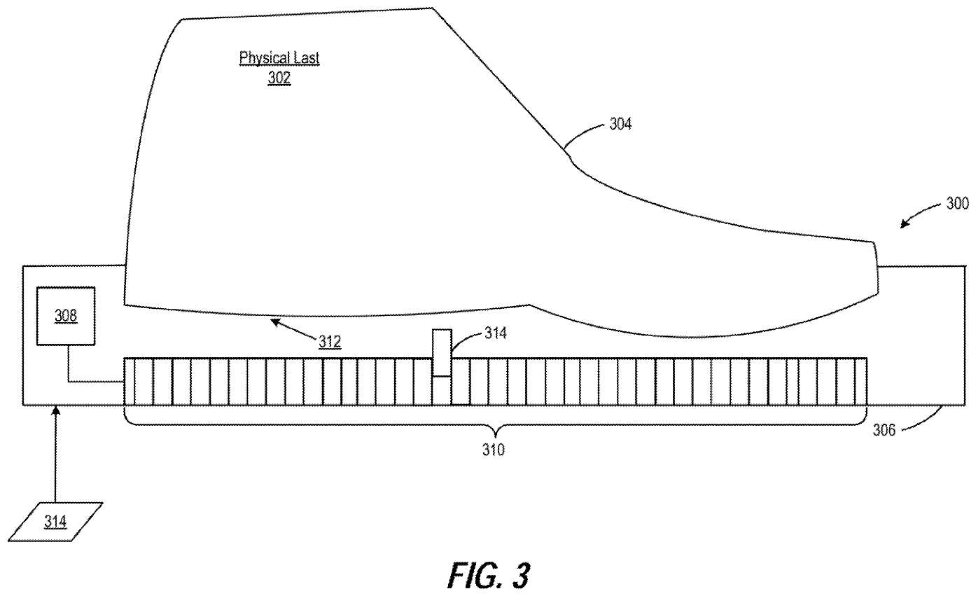

FIG. 3 illustrates a shaping device 300 that customizes a physical last 302 based on customer data to manufacture a component of a shoe, according to an example. Physical last 302 may include a housing 304. Shaping device 300 may include a housing 306, a controller 308, and a set of actuators 310. Controller 308 may be a central processing unit (CPU), a semiconductor-based microprocessor, and/or other hardware devices suitable to control set of actuators 310. Housing 306 may include a recessed region to encompass a region of housing 202 of physical last 302. For example, housing 306 may include a recessed region to encompass a bottom region 312 of housing 304. Housing 304 may be made from deformable material. For example, the deformable material may be high temperature silicone rubber.

During operation, shaping device 300 may receive shaping information 314 that defines a shape of bottom region 312. Shaping information 314 may be determined using 3D scan data 104 and/or pressure data 106. Based on shaping information 314, controller 308 may control a deployment of set of actuators 310. As an example, controller 308 may activate an actuator 316 of set of actuators 310 to define the shape of bottom region 312 by deforming a portion of bottom region 312. When the shape of bottom region 312 is defined, shaping device 300 may be detached from housing 304. Shaped physical last 302 may be used to manufacture a component of a shoe that is customized for a particular customer.

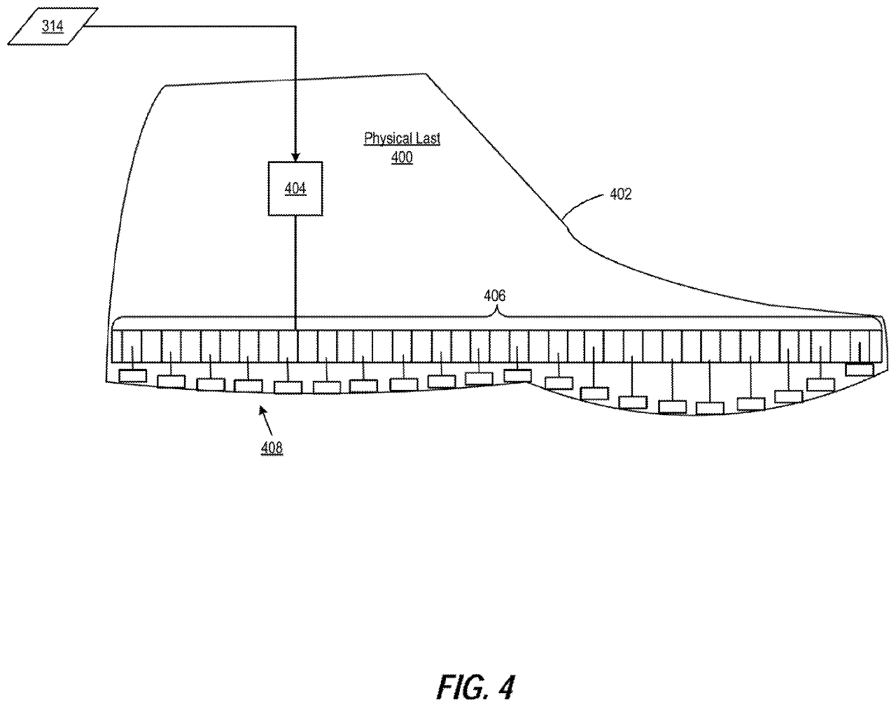

FIG. 4 illustrates a programmable physical last 400 to manufacture a component of a shoe based on customer data, according to an example. Physical last 400 may include a housing 402, a controller 404, and a set of actuators 406. Housing 402 may be similar to housing 304 of FIG. 3. Controller 404 may be a central processing unit (CPU), a semiconductor-based microprocessor, and/or other hardware devices suitable to control operations of set of actuators 406. During operation, controller 404 may receive shaping information 314. Based on shaping information 314, controller 404 may deploy set of actuators 406 to define a shape of a bottom region 408 of housing 402. When the shape of bottom region 408 is defined, shaped physical last 400 may be used to manufacture a component of a shoe that is customized for a particular customer.

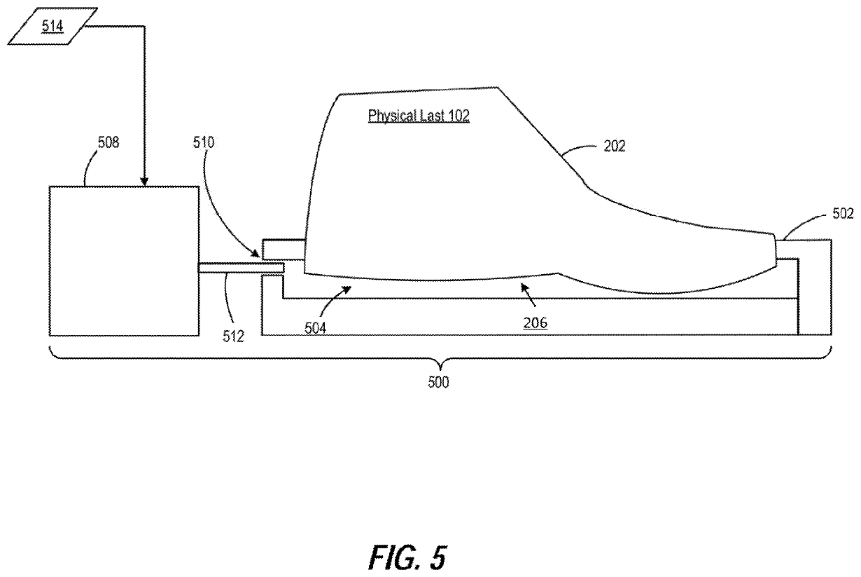

FIG. 5 illustrates an injection molding process to be used with a physical last to manufacture a component of a shoe based on customer data, according to an example. FIG. 5 is described in reference to physical last 102. However, it should be understood that any of physical lasts 302 and 404 may also be used in the molding process described in FIG. 5.

During a molding process, such as an injection molding process, a molding device 500 may be used to form a component of a shoe using physical last 102. Molding device 500 may include a housing 502 may include a cavity 504 that receives a portion of housing 202 to define a mold. For example, a portion of 202 may be encompassed by housing 502 such that bottom region 206 of housing 202 is encompassed by cavity 504. Thus, remaining space in cavity 504 (not taken up by housing 202) may define a mold for a component of a shoe. Molding device 500 may also include an injection machine 508. Housing 502 may include an opening 510 to receive a tip 512 of injection machine 508.

Injection machine 508 may determine how a component of a shoe is formed based on injection instructions 514. Injection instructions 514 may be determined based on 3D scan data 104 and/or pressure data 106. For example, injection machine 508 may, based on injection instructions 514, vary the speed and/or pressure of the material injected over time to change a property of the material. Thus, a first region of a component may be formed using a material having a first property. A second region of the component may be formed using the material with a second property different from the first property. The resulting component may have material properties that vary along a gradient depending on the speed and/or pressure of the material injected. As another example, injection machine 508 may inject different materials to form the component. Injection machine 508 may inject a first material during a first time period to form a first region of the component. Injection machine 508 may inject a second material during a second time period to form a second region of the component. Thus, the resulting component may be formed using different materials.

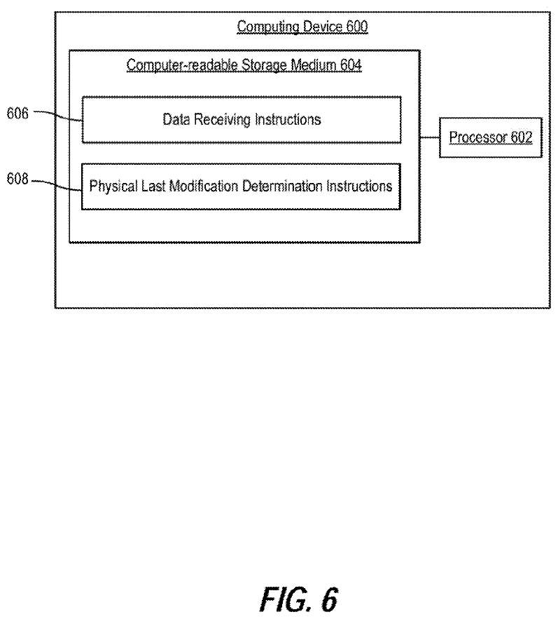

FIG. 6 illustrates a computing device 600 that determines customizations of a physical last based on customer data, according to an example. Computing device 600 may be used with any of system 100 of FIG. 1, shaping device 204 of FIG. 2, shaping device 300 of FIG. 3, and physical last 400 of FIG. 4.

Computing device 600 may include a processor 602 and a computer-readable storage medium 604. Processor 602 may include a central processing unit (CPU), a semiconductor-based microprocessor, and/or other hardware devices suitable to control operations of computing device 600. Computer-readable storage medium 604 may be any electronic, magnetic, optical, or other physical storage device that contains or stores executable instructions. Thus, computer-readable storage medium 604 may be, for example, Random Access Memory (RAM), an Electrically Erasable Programmable Read-Only Memory (EEPROM), a storage device, an optical disc, etc. In some examples, computer-readable storage medium 604 may be a non-transitory storage medium, where the term “non-transitory” does not encompass transitory propagating signals. As described in detail below, computer-readable storage medium 604 may be encoded with a series of processor executable instructions 606 and 608.

Data receiving instructions 606 may receive 3D scan data, such as 3D scan data 104, and/or pressure data, such as pressure data 106. Physical last customization determination instructions 608 may determine a modification to be applied to a physical last based on the 3D scan data and/or the pressure data received using data receiving instructions 606. For example, the modification may include dimensions of shaping device 204. As another example, the modification may include a digital representation of shaping device 204. As another example, the modification may include shaping information 314.

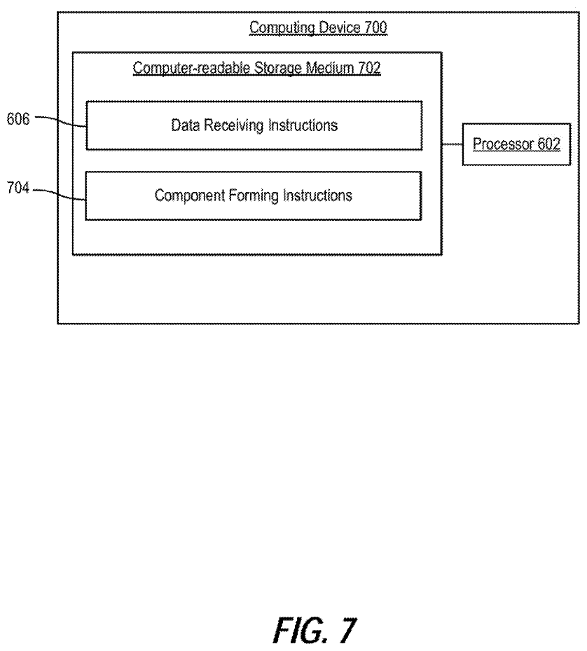

FIG. 7 illustrates a computing device 700 that determines customizations of a component of a shoe based on customer data, according to an example. Computing device 700 may be used during a molding process, such as with injection machine 508. Computing device 700 may include processor 602 and a computer-readable storage medium 702. Computer-readable storage medium 702 may be similar to computer-readable storage medium 604 of FIG. 6. Computer-readable storage medium 702 may be encoded with instructions 606 and 704. Component forming instructions 704 may determine how a component of a shoe is formed. For example, referring to FIG. 5, component forming instructions 704 may generate injection instructions 514 based on 3D scan data 104 and/or pressure data 106.

The use of “comprising”, “including” or “having” are synonymous and variations thereof herein are meant to be inclusive or open-ended and do not exclude additional unrecited elements or method steps.

What is claimed is:

1. A physical last comprising:
a body having a bottom region; and
a fitting attachable to the bottom region to define a component of a shoe, wherein the fitting is manufactured based on three-dimensional (3D) scan data of a foot of a customer and pressure data associated with the foot of the customer.

2. The physical last of claim 1, wherein the 3D scan data is generated via a 3D foot scanner.

3. The physical last of claim 1, wherein the pressure data is generated via a pressure sensor.

4. The physical last of claim 1, wherein the fitting is generated via a subtractive manufacturing process, an additive manufacturing process, a molding process, or a combination thereof.

5. The physical last of claim 1, wherein the component includes a midsole of a shoe, an insole of a shoe, or a combination thereof.

6. The physical last of claim 1, wherein the component includes an upper of a shoe.

7. A method comprising:
defining, via a shaping device, a shape of a bottom region of a physical last based on three-dimensional (3D) scan data of a foot of a customer and pressure data associated with the foot of the customer; and
forming, via a molding device attachable to the physical last, a component of a shoe using the shaped physical last.

8. The method of claim 7, wherein the shaping device includes to a fitting, and wherein forming the shape includes attaching the fitting to the bottom region.

9. The method of claim 8, wherein the fitting is generated based on based on the 3D scan data and the pressure data.

10. The method of claim 7, wherein the shaping device includes:
a housing encompassing the bottom region; and
a set of actuators deployable to define the shape of the bottom region, wherein a deployment of the set of actuators is determined based on the 3D scan data and the pressure data.

11. The method of claim 7, further comprising:
using, via the molding device, a first material to form a first region of the component; and
using a second material to form a second region of the component, wherein the first material and the second material are determined based on the 3D scan data and the pressure data.

12. The method of claim 7, further comprising:
using, via the molding device, a material to form a first region of the component;
changing a property of the material based on the 3D scan data and the pressure data; and
using the material with the changed property to form a second region of the component.

13. A non-transitory computer readable storage medium comprising instructions that when executed cause a processor of a computing device to:
receive, at the computing device, three-dimensional (3D) scan data of a foot of a customer and pressure data associated with the foot of the customer; and
determine, based on the 3D scan data and the pressure data, a modification to be applied to a physical last, wherein the modified physical last is to define a component of shoe.

14. The non-transitory computer readable storage medium of claim 13, wherein the modification includes a digital representation of a fitting attachable to the physical last.

15. The non-transitory computer readable storage medium of claim 13, wherein the modification includes instructions to determine a deployment of a set of actuators located in the physical last, and wherein the deployment of the set of actuators is to define the component.
